(19) United States
(12) Patent Application Publication
(10) Pub. No.: US 2020/0143923 A1
(43) Pub. Date: May 7, 2020

(54) CONTROLLED SUBSTANCE MISUSE PREVENTION

(71) Applicant: PROOF RX, LLC, Tallahassee, FL (US)

(72) Inventor: Russell Bennett Walther, Tallahassee, FL (US)

(21) Appl. No.: 16/458,983

(22) Filed: Jul. 1, 2019

Related U.S. Application Data
(60) Provisional application No. 62/755,536, filed on Nov. 4, 2018.

(51) Int. Cl. G16H 20/10

(52) U.S. Cl. CPC G16H 20/10

(57) ABSTRACT

Systems and methods are described herein for determining a therapeutic use date after which consumption of a prescribed medication by a legitimately prescribed individual will provide little to no legitimate therapeutic value to the individual. By determining the therapeutic use date and then marking each unit of the individual's prescription with an indication of the therapeutic use date, the systems and methods described herein aid in the regulation or removal of unused controlled substances from the individual after the therapeutic use date.

### RELATED APPLICATIONS

This application claims priority to U.S. Provisional Patent Application No. 62/755,536, filed Nov. 4, 2018, entitled “Controlled Substance Misuse Prevention Device and Method,” which is hereby incorporated by reference herein in its entirety.

### FIELD

The present disclosure relates to combating the prescription drug overdose epidemic, and more specifically is related to associating units of medication with a date by which consumption of the medication by an individual will have little to no legitimate therapeutic value to the individual.

### BACKGROUND

The misuse of and addiction to prescription drugs—including prescription opioids, central nervous system depressants, and stimulants—is a serious national crisis that affects public health as well as social and economic welfare. Although most people may take prescription medications responsibly, according to the National Institute on Drug Abuse, an estimated 18 million people had misused such medications at least once in 2016. The reasons for the high prevalence of prescription drug misuse vary by age, gender, and other factors, but likely include the absence of a widespread practice to remove unused controlled substances from a legitimately prescribed user. Too often, the legitimately prescribed user ends up with leftover medication that is ultimately misused, for example by the legitimately prescribed user himself, or a friend, relative, thief, or other.

### SUMMARY

Some embodiments of the present disclosure provide for associating units of medication with a therapeutic use of the medication by an individual. A method for associating units of medication with a therapeutic use of the medication by an individual can include identifying prescription information associated with an individual and a prescription for a plurality of units of medication; determining a therapeutic use date to be added to each of the plurality of units of the medication based at least in part on the prescription information; and causing an apparatus to add the therapeutic use date to each of plurality of units of the medication. The therapeutic use date can be different from and later than a strict adherence date associated with the prescription. The therapeutic use date can be different from and earlier than an expiration date of the plurality of units of the medication.

The method of the preceding paragraph may also include any combination of the following steps or features described in this paragraph, among other steps of features described herein. The therapeutic use date can correspond to a date after which consumption of any of the plurality of units of the medication will have no legitimate therapeutic value to the individual. The therapeutic use date can correspond to a date after which the amount of therapeutic value provided to the individual when consuming any of the plurality of units of the medication does not satisfy a threshold therapeutic value. The therapeutic use date can correspond to a date by which the individual should no longer be in possession of any of the plurality of units of the medication. The therapeutic use date can correspond to a date after which the individual should dispose of any unused ones of the plurality units of the medication. The therapeutic use date can correspond to a date after which the individual return any unused ones of the plurality units of the medication to a medication-return facility.

The method of any of the preceding paragraphs may also include any combination of the following steps or features described in this paragraph, among other steps of features described herein. The medication can be at least one of a prescription opioid, central nervous system depressant, or stimulant. The prescription information can include at least a number units of the medication in the prescription and a dosing schedule associated with the prescription. The dosing schedule can indicate an amount of time between doses of the medication. Said determining the therapeutic use date can include: multiplying the number units of the medication in the prescription by the amount of time between doses of the medication to determine a minimum amount of time the prescription can be completed according to the dosing schedule; selecting a base multiple based at least in part on the prescription information; and multiplying the base multiplier by the minimum amount of time the prescription can be completed according to the dosing schedule.

The method of any of the preceding paragraphs may also include any combination of the following steps or features described in this paragraph, among other steps of features described herein. The therapeutic use date can be extrapolated based at least in part on a date of the prescription and said multiplying the base multiplier by the minimum amount of time the prescription can be completed according to the dosing schedule. The base multiplier can be an individual-specific base multiplier. The base multiplier can be based at least in part on a historical use of the medication by the individual. The strict adherence date can correspond to a minimum amount of time the prescription can be completed according to the dosing schedule. Each of the plurality of units of the medication can include at least one of a pill, tablet, or capsule. The expiration date can be based at least in part on at least one of a chemical efficacy of the prescribed medication.

Some embodiments of the present disclosure provide a system for associating units of medication with a therapeutic use of the medication by an individual. The system can include one or more processors. The one or more processors can be configured to identify prescription information associated with an individual and a prescription for a plurality of units of medication. The one or more processors can be further configured to determine therapeutic use date to be added to each of the plurality of units of the medication based at least in part on the prescription information. The therapeutic use date can be different from and later than a strict adherence date associated with the prescription. The therapeutic use date can be different from and earlier than an expiration date of the plurality of units of the medication. The one or more processors can be further configured to cause an apparatus to add the therapeutic use date to each of plurality of units of the medication.

The system of the preceding paragraph may also include any combination of the following features described in this paragraph, among other features described herein. The therapeutic use date can correspond to a date after which consumption of any of the plurality of units of the medication will have no legitimate therapeutic value to the individual. The therapeutic use date can correspond to a date by which the individual should no longer be in possession of any of the plurality of units of the medication. To determine the therapeutic use date, the one or more processors can be further configured to: multiply a number units of the medication in the prescription by an amount of time between doses of the medication to determine a minimum amount of time the prescription can be completed according to a dosing schedule; select a base multiple based at least in part on the prescription information; and multiply the base multiplier by the minimum amount of time the prescription can be completed according to the dosing schedule. The base multiplier can be an individual-specific base multiplier. The base multiplier can be based at least in part on a historical use of the medication by the individual. The strict adherence date can correspond to a minimum amount of time the prescription can be completed according to the dosing schedule.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 is a block diagram of an example of an environment for associating individual units of medication with a therapeutic use of the medication by an individual.

FIG. 2 is a flow diagram illustrative of an embodiment of a routine for determining a therapeutic use date for a medication prescribed to an individual.

FIG. 3 illustrates example units of medication that have been marked with an indication of a therapeutic use date.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

Despite the prescription drug epidemic, legitimately prescribed users are regularly prescribed more medication than they actually use. In some cases, the leftover medication is a result of overprescribing—where a provider prescribes an excessive amount of the medication to an individual. In other cases, the legitimately prescribed users fail to finish the prescription, for example by failing to take the full daily dose or otherwise failing to finish all of the pills that are prescribed. Too often, when a legitimately prescribed user improves, he stops taking his prescription medication. In such cases, the excess prescription medications can end up in a medicine cabinet, in hands of friends or family members, or sold, for example, because of the medication's high resale value in a sea of seemingly endless customers.

To date, there is no effective, widespread practice to remove unused controlled substances from the end-user. Furthermore, there is no practice in place for holding an individual accountable for the whereabouts of his own prescription medications. These shortcomings are enhanced, at least in part, by that fact that once a pill leaves its medical container, a visual inspection of the pill does not provide information as to whom the pill was prescribed, the date the pill was prescribed, or its medication contents. Thus, if the medical container is emptied and lost or otherwise discarded, unused pills are unidentifiable, particularly if the pill lacks any brand name identification.

Due to the above and other shortcomings, there is a general need for improved techniques and technologies for removing unused controlled substances from the end-user. Systems and methods are described herein for determining a therapeutic use date of the medication and adding an indication of the therapeutic use date to each unit of medication. Although various aspects of the disclosure will be described with regard to examples and embodiments, one skilled in the art will appreciate that the disclosed embodiments and examples should not be construed as limiting.

In the present disclosure, reference is made to medication. As used herein, the term “medication” is used broadly to refer to any drug that is distributed from a pharmacy or otherwise used in the medical treatment of an individual. By way of non-limiting example, a medication can include a drug prescribed by a provider that is intended for use to diagnose, cure, treat, or prevent disease, pain, ailments, illness, symptoms, or the like. In some cases, a medication is a pharmaceutical drug that legally requires a medical prescription to be dispensed, such as a prescription opioid. Medication can come in many forms, such as, but is not limited to, liquids, tablets, capsules, topical medicines, suppositories, drops, inhalers, implants, or patches.

In some cases, the term “medication” is used broadly to refer to a controlled substance, or any one or more schedules of controlled substances. For example, in some cases, a medication can include a Schedule II or Schedule IIN narcotic such as, but not limited to, hydromorphone (Dilaudid), methadone (Dolophine), meperidine (Demerol), oxycodone (OxyContin, Percocet), fentanyl (Sublimaze, Duragesic), morphine, opium, codeine, amphetamine (Dexedrine, Adderall), methamphetamine (Desoxyn), methylphenidate (Ritalin), amobarbital, glutethimide, or pentobarbital. In some cases, a medication can include a Schedule III narcotic such as, but not limited to, combination products containing less than 15 milligrams of hydrocodone per dosage unit (Vicodin), products containing not more than 90 milligrams of codeine per dosage unit (Tylenol with Codeine), or buprenorphine (Suboxone). In some cases, a medication can include a Schedule IIIN non-narcotic such as, but not limited to, benzphetamine (Didrex), phendimetrazine, ketamine, and anabolic steroids such as Depo-Testosterone. In some cases, a medication can include a Schedule IV substance such as, but not limited to, alprazolam (Xanax), carisoprodol (Soma), clonazepam (Klonopin), clorazepate (Tranxene), diazepam (Valium), lorazepam (Ativan), midazolam (Versed), temazepam (Restoril), and triazolam (Halcion). In some cases, a medication can include a Schedule V substance such as, but not limited to, cough preparations containing not more than 200 milligrams of codeine per 100 milliliters or per 100 grams (Robitussin AC, Phenergan with Codeine), and ezogabine. In some cases, a medication can include a Schedule I substance such as, but not limited to, heroin, lysergic acid diethylamide (LSD), marijuana (cannabis), peyote, methaqualone, or 3,4-methylenedioxymethamphetamine (“Ecstasy”).

Throughout this disclosure, examples include medications described in the form of units. In the present disclosure, reference to the term “unit” is not to be construed as limiting and should to refer to any portion of a medication. By way of non-limiting example, a unit of medication can include a single dose or multiple doses of the medication. As another non-limiting example, the term “unit” of medication can include an individual pill, tablet, or capsule of the medication.

The term “prescription” is used broadly herein to refer to any instruction written, or electrically entered, by a medical practitioner that authorizes an individual to be provided a medication. A prescription can refer to any order, list, label, etc., that includes one or more units of medication. In some cases, a doctor or other healthcare professional can generate the prescription. As one example, a prescription can be a list of medications to be dispensed to and taken by an individual. In some instances, the prescription can specify a particular dose of the medication to be taken by the individual. In some cases, the prescription can specify particular dosing schedules, including, for example, when, how, or how frequently to take the medication. In some cases, the prescription can provide an indication of a name or other information corresponding to the individual for whom the prescription was written.

The term “expiration date” is used herein to refer to a date after which the medication might not be suitable for use as manufactured. For purposes of this disclosure, an expiration date is not necessarily an indication of when the medication will become ineffective or unsafe to use. Rather, in some cases, the expiration date corresponds to a manufacturer's promise for a time until which the medication will have full efficacy and be safe as manufactured. In some cases, the expiration date corresponds to a date estimated using stability testing under good manufacturing practices as determined by the Food and Drug Administration.

The term “strict adherence date” is used herein to refer to a date after which an individual's supply of the medication will be exhausted if the individual strictly adhered to a dosing schedule indicated by a medical profession, such as one indicated in the prescription. For example, if an individual is prescribed 20 units of a medication, one unit to be taken per day, then the strict adherence date can correspond to 20 days after the medication is dispensed to the individual because the dosing schedule is one unit per day for 20 days.

The term “therapeutic use date” is used herein to refer to a date after which consumption of the medication by the individual will have little to no legitimate therapeutic value to the individual. In other words, the therapeutic use date is based on anticipated resolution of the individual condition for which the prescription was written. In general, the therapeutic use date is different from strict adherence date. For instance, an individual may be prescribed 20 units of a medication, one unit to be taken per day. However, after 20 days, the individual may have mistakenly (for example, the individual forgot) or intentionally (for example, the individual was feeling better that day) missed one or more days, such that at the time of the strict adherence date, the individual continues to have some of the units of the medication in his possession. Even though the strict adherence date has passed, in some cases, taking some or all of these remaining pills after the strict adherence date may continue to provide the individual with a therapeutic value. Furthermore, in general, the therapeutic use date is different from, and earlier than, the expiration date. For example, especially in instances in which the individual takes the medication to alleviate pain from a surgery or injury, the individual is likely to recover from the condition for which the prescription was written (thus, leaving little to no therapeutic value of the medication) prior to the expiration date of the medication.

The therapeutic use date is generally a date that falls sometime between the strict adherence date and the expiration date, although, in some cases, the therapeutic use date is the same or earlier than the strict adherence date. As described herein, the therapeutic use date can be determined electrically via a computing device or manually. In some cases, the therapeutic use date can be individual-specific. For example, the therapeutic use date for a particular medication for a particular individual can be based at least in part on the type or prescribed dosage of the medication, the reason for the prescription (for example, the condition for which the prescription was written), the individual's medical history (for example, the length of time the individual previously took to recover from the same or a similar condition), medical history of other individuals (for example, the length of time similarly situated individuals took to recover from the same or a similar condition), etc.

As a non-limiting example, in some cases, a prescription is entered or generated by a doctor or other healthcare professional using a computing device. In some instances, the prescription can be manually created, for example, handwritten, and later entered into a computerized system. Following entry into the computerized system, the prescription is released to be filled, or otherwise prepared. The prescription can be filled either manually, for example, by a pharmacist or other pharmacy technician, or automatically, for example, by an automated pill picker or drug carousel. A therapeutic use date can be determined, for example, based at least in part on the individual's condition, the prescribed medication, a dosing schedule, or the like, and an indication of the therapeutic use date can be added to each unit of medication to be provided to the individual. Once the prescription is filled and the therapeutic use date is added to the units of medication, the prescription can be dispensed or delivered to the individual.

Marking the therapeutic use date individual units of medication advantageously improves the likelihood that unused medications will not be abused. For example, in some cases, the legitimately prescribed user may be obligated to use, destroy, or return, any medication unit that is unused at the time of the therapeutic use date. Notably, because the therapeutic use date corresponds to a time in which consumption of the medication by the legitimately prescribed user will have little to no legitimate therapeutic value to the legitimately prescribed user, the legitimately prescribed user is able to obtain all or most of the therapeutic benefits from the prescribed medication. Furthermore, in effect, the systems and methods herein turn the individual units of medication into a time-delineated license in that the legitimately prescribed user should use, destroy, or return any remaining units of medication on or before the therapeutic use date.

The methods and systems described herein can allow for increased accountability of the individual units of medication after the therapeutic use date. In some instances, the legitimately prescribed user can be legally required to use, destroy, or return the unused units of medication after the expiration of therapeutic use date. For example, in some cases, it can be a criminally punishable offense to possess any units of medication after the expiration of the therapeutic use date. Similarly, in some cases, it can be a criminally punishable offense to possess any units of medication in which the therapeutic use date has been removed or altered. In some cases, in addition to an indication of the therapeutic use date, each unit of medication can include an individual identifier. In cases such as these, it can be a criminally punishable offense for an individual to possess any unit of medication that has an individual identifier corresponding to a different individual.

In some cases, the units of medication are to allow end-user identification with a specific prescription encounter, and to allow a prescriber/dispenser to place time delineated information on the medication unit. For example, a marked unit of medication can allow for visual identification of the individual to which the medication is prescribed or the time of a prescriber/dispenser delineated action to that individual medication unit. This can ultimately lead to appropriate handling of unused medication that would otherwise be available for misuse. For example, the individual identification of each medication unit allows for traceability. If a medication unit is traceable, then any of the following scenarios are possible: depletion of the medication in routine legitimate use; securing unused medication for future legitimate use; destruction of the medication after need has passed in legitimate use; legitimate loss of the medication; illicit sale of the medication; or legitimate theft of the medication.

Systems and methods for determining a therapeutic use date or adding the therapeutic use date to individual units of medication will now be described in greater detail, by way of example, in reference to several embodiments shown in the figures.

### Overview of Example Environment

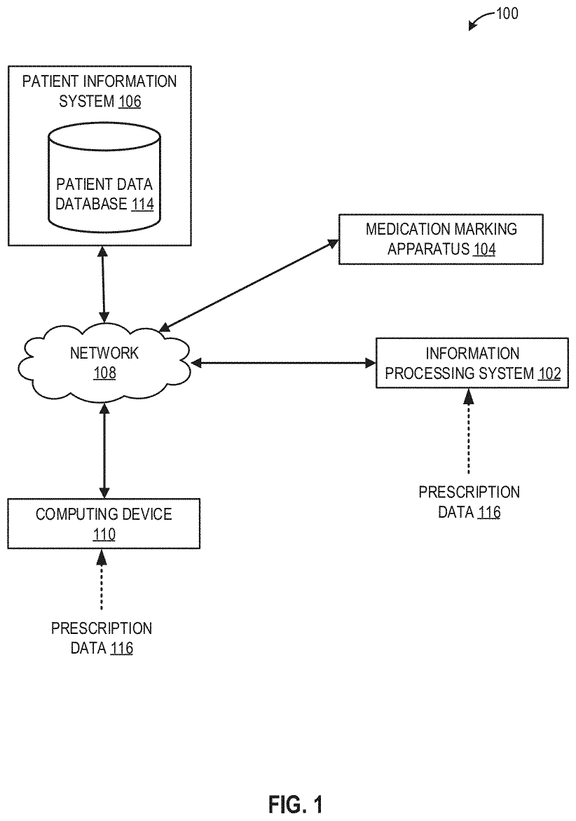

FIG. 1 is a block diagram illustrating an environment 100 for determining a therapeutic use date of one or more individual units of a medication or adding the therapeutic use date to one or more individual units of the medication. In the illustrated embodiment, the environment 100 includes an information processing system 102, a medication marking apparatus 104, a patient information system 106, a computing device 110, and a network 108. However, it will be understood that fewer or more devices can be used as desired.

Any of the foregoing systems or apparatuses of environment 100 may communicate via a network 108. Although only one network 108 is illustrated, multiple distinct and/or distributed networks 108 may exist. The network 108 can include any type of communication network. For example, the network 108 can include one or more of a wide area network (WAN), a local area network (LAN), a cellular network, an ad hoc network, a satellite network, a wired network, a wireless network, and so forth. In some embodiments, the network 108 can include the Internet. Furthermore, although illustrated as being in communication via the network 108, in some implementation, two or more of the foregoing systems or apparatuses may communicate without using the network 108.

In certain embodiments, some or all of the information processing system 102, the medication marking apparatus 104, the patient information system 106, or the computing device 110 can form part of a single device and communicate via a computer bus. Regardless of the configuration, each separate device can include one or more processors or controllers and a computer-readable medium that stores specific computer-executable instructions for carrying out the operations described herein.

The information processing system 102 can be configured to determine, retrieve, or identify a therapeutic use date for a unit of medication. For example, in some cases, the information processing system 102 can communicate with the patient information system 106 or the computing device 110 to retrieve or receive data to be used to determine the therapeutic use date. In addition or alternatively, data, such as prescription data, can be input into the information processing system 102 and the information processing system 102 can use the data to determine a therapeutic use date.

The information processing system 102 may include hardware and software components for controlling the medication marking apparatus. For example, the information processing system 102 can communicate, for example over the network 108, with the medication marking apparatus 104 to cause the medication marking apparatus 104 to print, label, engrave, or otherwise mark the corresponding therapeutic use date, or indication thereof, onto each unit of medication to be dispensed to the individual. In some cases, the medication marking apparatus 104 can add identifiers other than a therapeutic use date identifier. For example, the medication marking apparatus 104 may be controlled to drill, print, label, engrave, press, imprint, or otherwise mark a medication identifier, such as a type or brand of the medication, or an individual identifier, such as an identifier that identifies the individual to which the medication was prescribed. In some instances, the information processing system 102 generates a file, which is sent to the medication marking apparatus 104. The medication marking apparatus 104 drills, prints, labels, engraves, presses, imprints, or otherwise marks the individual units of medication based on the file. As a non-limiting example, the medication marking apparatus can include one or more commercially available medication marking apparatuses, such as, but not limited to, the “Pill Mini Press Machine Lab Professional Tablet Manual Punching Machine” available from ZONESUN, the “Lab Scale Drilling Laser System” available from ScanTech Laser, the “Batch Submission Tablet Drilling Laser System” available from ScanTech Laser, the “High Speed Tablet Drilling Laser System” available from ScanTech Laser, the “Softsule Laser Marking Machine” available from ScanTech Laser, or the “High Speed Tablet Marking Laser System” available from ScanTech Laser.

The information processing system 102 may be equipped with networking equipment and network software applications (for example, a web browser) that facilitate communications via one or more networks (for example, the Internet or an intranet). The information processing system 102 may have varied local computing resources such as central processing units and architectures, memory, mass storage, graphics processing units, communication network availability and bandwidth, and so forth. Further, the information processing system 102 may include any type of computing system. For example, the information processing system 102 may include any type of computing device(s), such as desktops, laptops, television set-top boxes, televisions (for example, Internet TVs), and wireless mobile devices (for example, smart phones, PDAs, tablets, or the like), to name a few.

The information processing system 102 can be controlled by a user, such as a pharmacist at a pharmacy in which an individual is picking up a prescribed medication. Thus, in some cases, the information processing system 102 is utilized at the point-of-sale of the prescription.

The computing device 110 can be configured to provide data 116 relating to an individual's prescription or other information to the information processing system 102 or the patient information system 106. For example, the computing device 110 may be equipped with networking equipment and network software applications (for example, a web browser) that facilitate communications via one or more networks (for example, the Internet or an intranet). The computing device 110 may have varied local computing resources such as central processing units and architectures, memory, mass storage, graphics processing units, communication network availability and bandwidth, and so forth. Further, the computing device 110 may include any type of computing system. For example, the computing device 110 may include any type of computing device(s), such as desktops, laptops, television set-top boxes, televisions (for example, Internet TVs), and wireless mobile devices (for example, smart phones, PDAs, tablets, or the like), to name a few.

In some cases, the computing device 110 is a computing device used by a doctor or other healthcare professional to enter a prescription. For example, the prescription can be entered into the computing device 110 and some or all of the prescription data, or other data, can be provided to the information processing system 102 or the patient information system 106.

In some cases, the patient information system 106 can include a patient data database 114, such as a physician drug monitoring program database (PDMP). For example, the patient information system 106 can include certain demographics such as first name, last name, date of birth, age, weight, sex, or geographic region. In some cases, the patient information system 106 can include an identified individual and their history of medication use or medication abuse. For instance, data can includes who prescribed the medications, as well as type, volume, and duration of medication. In addition or alternatively, patient data database 114 can include the reason the individual was prescribed the medication, as well as the prescription data regarding the individual's medication, a length of time the individual took the medication, how long it took the individual to recover from the condition, etc.

In some cases, the prescription data 116 can be entered into the environment 100, for example via the computing device 100, the information processing system 105, or the patient information system 106. The prescription data can include, but is not limited to, a identifier of the prescribing practitioner (for example, a name, the practitioner's federal Drug Enforcement Administration (DEA) registration number, the practitioner's National Provider Identification (NPI) or other appropriate identifier), an individual identifier (for example, a name, address, telephone number, or date of birth) of the person for whom the prescription was written, a medication identifier (for example, a name, a national drug code, a quantity, or a strength of the medication), the date of the prescription, the date the prescription was filled, the method of payment, the full name from which the medication was dispensed, the federal Drug Enforcement Administration registration number from which the medication was dispensed, the address of the pharmacy or other location from which the medication was dispensed, the name of the dispensing pharmacy, the name of the dispensing practitioner, other than a pharmacist, the dispensing pharmacy or practitioner National Provider Identification number, any portion of other appropriate identifying information as determined by department rule, drug class, etc. In some cases, the prescription data 116 can include any data in the patient information system 106.

### Flow Diagrams

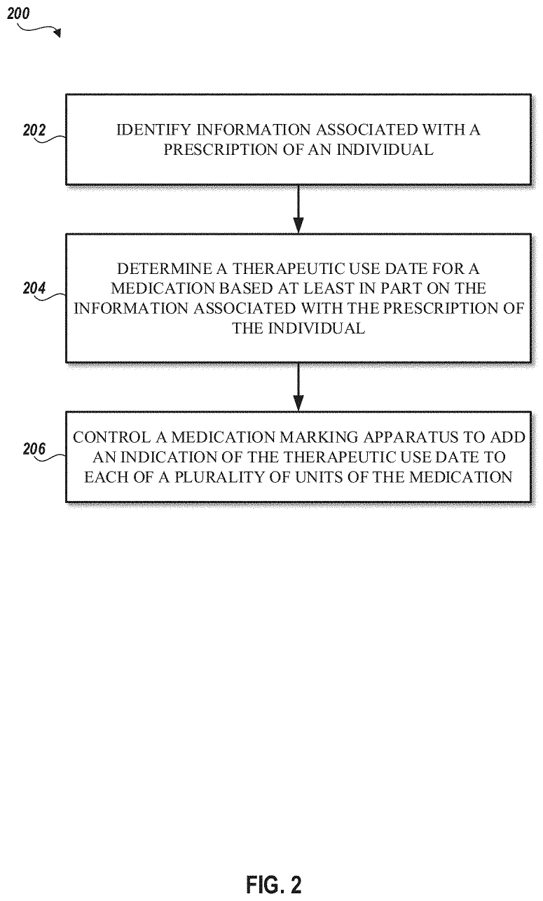

FIG. 2 is a flow diagram illustrative of an embodiment of a routine 200 for determining a therapeutic use date for a medication prescribed to an individual. One skilled in the relevant art will appreciate that the elements outlined for routine 200 can be implemented by one or more computing devices that are associated with the environment 100, such as the information processing system 102. Although any number of systems, in whole or in part, can implement the process 200, to simplify discussion, the routine 200 will be described as being generally performed by the information processing system 102. However, the following illustrative embodiment should not be construed as limiting.

At block 202, the information processing system 102 identifies or obtains information associated with an individual's prescription. For example, as described herein, the information processing system 102 can communicate with a computing device or a patient information system 106 to obtain the information associated with the individual's prescription. In addition or alternatively, the information associated with the individual's prescription can be input into the information processing system 102, for example by a pharmacist at the pharmacy at which the individual is filling the prescription.

The information that the information processing system 102 identifies or obtains at block 202 can vary across embodiments. For example, in some cases, the information can include data that the information processing system 102 can utilize to calculate or determine a therapeutic use date for the medication associated with the individual's prescription.

As an example, the information can include a prescribed dose, which can indicate the amount of the medication to be taken by the individual at any one time. The prescribed dose can be can be expressed as a weight of medication (for example, 250 mg), volume of medication (for example, 10 mL, 2 drops), the number of dosage forms (for example, 1 capsule, 1 suppository) or some other quantity (for example, 2 puffs).

As another example, the information can include a prescribed dosage regimen, which can include the frequency at which the medication doses are intended to be taken by the individual. Examples include 2.5 mL twice a day, one tablet three times a day, one injection every four weeks. In some cases, the information can include a total daily dose, which can be calculated from the prescribed dose and the dosage regimen.

As another example, the information can include individual information. For instance, the individual information can include height, sex, weight, age, or other an individual identifier that identifies the individual or one or more characteristics of the individual. In some cases, individual information can include information regarding the individual's condition, such as the disease, pain, ailments, illness, symptoms, injury, or surgery that relates to the reason for the prescription, or a projected recovery time associated with the individual's condition. In some cases, the projected recovery time can be based at least in part on a previous recovery time by the individual for the same or a similar condition or a previous recovery time by one or more other similar situated individuals (for example, age, sex, height, weight, or the like) for the same or a similar condition.

As another example, the information can include one or more dates, such as an expiration date of the medication or a strict adherence date associated with the individual's prescription.

At block 204, the information processing system 102 determines a therapeutic use date for the prescribed medication based at least in part on the information that the information processing system 102 identified or obtained at block 202. As described herein, the therapeutic use date refers to a date or time after which consumption of the medication by the individual will have little to no legitimate therapeutic value to the individual. Thus, the therapeutic use date serves as a reminder of the date by which the individual should no longer possess the medication, or at least of the date after which the individual should consume, dispose or, or return the medication.

The information processing system 102 can determine the therapeutic use date for a particular unit of medication using one or more of various techniques. For example, in some cases, the therapeutic use date can be determined by adding a particular amount of days to strict adherence date, which can act as a buffer to the strict adherence date to allow the individual additional time to finish the prescription in case the individual misses doses. The additional amount of time that is added to the strict adherence date can vary across embodiments. For example, the additional amount of time can be a set period of time, such as one, two, three, four, five, or six days, weeks, months, or years. As another example, the additional amount of time can be a function related to the strict adherence date. For example, the period between the date of the prescription and the strict adherence date can be referred to as the strict adherence period. In some cases, the additional amount of time can be a multiplier of the strict adherence period. For example, the additional amount of time can correspond to 0.1, 0.2, 0.25, 0.5, 0.8, 1, 2, 2.5, or 3 times the number of days or hours of the strict adherence period.

As another example, in some cases, the therapeutic use date can be based at least in part on a past patient data related to the individual, or comparable patient data related to one or more other individuals. For example, assume the individual is having a knee replacement surgery on his left knee, after having previously has a knee replacement surgery on his right knee. In some cases, the information determined by the information processing system 102 at step 202 includes an amount of time (for example, an estimated or exact amount of time) that the individual took medication for the first knee replacement surgery. For instance, the information processing system 102 can determine an expected date that the individual will no longer need, use, or want to take medication based on the first knee replacement surgery, and the therapeutic use date can correspond to that expected date. Similarly, the information processing system 102 can use comparable patient data from other individuals to estimate an expected date that the individual will no longer need, use, or want to take medication, and the therapeutic use date can correspond to that estimated date.

As another example, the therapeutic use date can be based at least in part on the prescribed medication. For example, each medication may be part of a particular drug class. In some cases, the therapeutic use date is based at least in part on the drug class of the prescribed medication and/or the volume or dose of the prescription.

As another example, the therapeutic use date can be based at least in part on the condition for which the medication is prescribed. For instance, chronic pain management individuals may be assigned a later therapeutic use date than those individuals that suffer from acute pain, because it can be assumed that the medication will have a longer legitimate therapeutic use for the individual with chronic pain than the individual with acute pain.

At block 206, the information processing system 102 controls the medication marking apparatus to add the therapeutic use date, or an indication thereof, to each of the units of medication before the units of medication are dispenses to the individual. For example, the information processing system 102 can communicate, for example over the network 108, with the medication marking apparatus 104 to cause the medication marking apparatus 104 to drill, print, label, engrave, press, imprint, or otherwise mark the corresponding therapeutic use date, or indication thereof, onto each unit of medication to be dispensed to the individual.

It will be understood that some or all of the various blocks described herein with reference to FIG. 2 can be implemented concurrently or have the order changed as desired. Furthermore, it will be understood that fewer, more, or different blocks can be used as part of the routine 200. For example, in some embodiments, the medication marking apparatus 104 can be controlled to add identifiers in addition to, or in place of, a therapeutic use date identifier, such as an individual identifier that identifies the individual to which the medication was prescribed.

### NON-LIMITING EXAMPLES

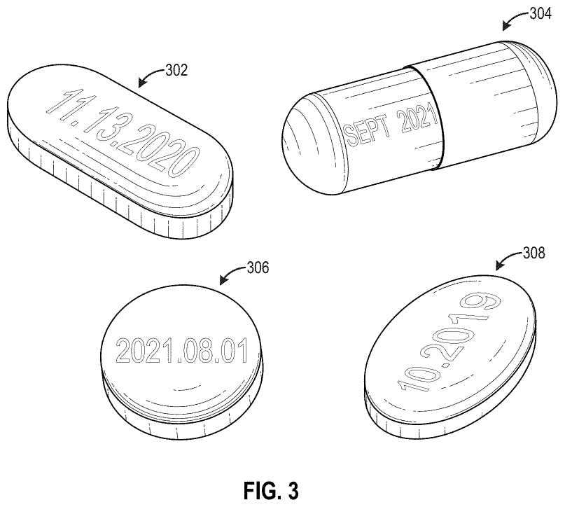

FIG. 3 illustrates example units of medication that have been marked with an indication of a therapeutic use date. As illustrated by the four units of medications 302, 304, 306, and 308 displayed in FIG. 3, the units of medication can be various shapes, sizes, materials, etc. Furthermore, FIG. 3 demonstrates that the units of medication can be marked using various techniques, such drilling, printing, labeling, engraving, pressing, imprinting, or otherwise marking. In addition, FIG. 3 demonstrates that the indication of the therapeutic use date can be added to the units of medication using different date formats. For example, unit 302 illustrates a Day-Month-Year format, unit 304 illustrates a Month-Year format, unit 306 illustrates a Year-Month-Day format, and unit 308 illustrates a Month-Year format. However, it will be understood that various date formats can be utilized, as well as various shapes and sizes. Furthermore, it will be understood that additional or different data can be added to a unit of medication, such as an identifier corresponding to the individual to which the medication was prescribed.

As a first non-limiting example, the environment 100 can be used to determine a therapeutic use date associated with an individual's prescription and then add that therapeutic use date to the units of medication corresponding to a prescription received by an individual from a physician. For example, the individual visits a healthcare professional, for example because of a condition of the individual, and the healthcare professional writes the individual a prescription by entering prescription data 116 into a computing device 110, such as the healthcare professional's laptop computer. In this example, the prescription is for 20 doses of a narcotic medication to be taken every 4-6 hours as needed for pain.

Following entry into the computerized system, the prescription or prescription data 116 is released, for example to the patient information system 114 or the information processing system 102. A pharmacist or other pharmacy technician retrieves the prescription data 116 and/or other information associated with the individual's prescription. For example, the pharmacist or other pharmacy technician can utilize the information processing system 102 to query the patient information system 106 for at least some of the prescription data 116 and/or other information associated with the individual's prescription. The prescription is filled or otherwise prepared. For example, the prescription can be filled either manually (non-limiting examples: by the pharmacist or other pharmacy technician) or automatically (non-limiting examples: by an automated pill picker or drug carousel).

In addition, the information processing system 102 determines a therapeutic use date using at least some of the prescription data 116 and/or other information associated with the individual's prescription. For example, based on the number of prescribed doses (in this case, 20) and the minimum time between doses (in this case, 4 hours), the information processing system 102 determines that the minimum amount of time to safely complete the prescription is 80 hours (for example, taking one dose every 4 hours). The information processing system 102 then determines the therapeutic use date by extending the minimum amount of time to safely complete the prescription (i.e., 80 hours) by a base multiplier (for example, 2, 4, 5, 8, 10, 15, 20). The base multiplier can be selected based at least in part on patient data (for example, age, sex, diagnosis code, weight, BMI, etc.).

In this example, a base multiplier of 8 is selected. Thus, to determine the therapeutic use date, the information processing system 102 multiplies the selected base multiplier (i.e., 8) by the minimum amount of time to safely complete the prescription (i.e., 80 hours) to calculate a number of hours (in this case, 640) between the prescription date and the therapeutic use date. The information processing system 102 divides the result by 24 to determine the number of days (in this case, 26.6) between the prescription date and the therapeutic use date. Assuming the date that the prescription is being filled is Jun. 1, 2019, the information processing system 102 can use the result of its calculations to determine the therapeutic use date of Jun. 27, 2019 (for example, June 1 plus 26.6 days).

After determining the therapeutic use date, the information processing system 102 controls the medication marking apparatus 104 to add an indication of the therapeutic use date to each units of medication being provided to the individual as part of the prescription. For example, in this case, the prescription corresponds to 20 individual tablets. Thus, the information processing system 102 can control the medication marking apparatus to add the indication of the therapeutic use date to each of the 20 tablets. Once the prescription is filled and the individual units of medication marked, the prescription can be dispensed or delivered to the individual.

As a second non-limiting example, a healthcare professional handwrites or prints a prescription for the individual and provides the prescription to the individual. The individual presents the prescription to a pharmacist or other pharmacy technician, and the pharmacist or other pharmacy technician enters prescription data 116 into the information processing system 102, such as a pharmacy computer. In this example, the prescription corresponds to 40 tablets, every 4-6 hours as needed, for a hip replacement on a 60 year old female patient.

The information processing system 102 determines a therapeutic use date using at least some of the prescription data 116 and/or other information associated with the individual's prescription. For example, based at least in part on the individual's age (i.e., 60), sex (i.e., female), or condition (i.e., recovering from hip surgery), the information processing system 102 can determine therapeutic use date based on how long similarly situated individuals took to recover. In this example, the information processing system 102 queries the patient information system 106 for information on similarly situation individuals and determines that 55-65 year old females patient with a hip replacement generally utilize only 13 tablets, and generally only take tablets for a period of 6 days. Based on this information, the information processing system 102 determines the therapeutic use date based on a base multiplier of the average period (in some cases, 6 days) of similarly situated individuals. For example, the base multiplier can be selected to be 1.5. Thus, to determine the therapeutic use date, the information processing system 102 multiplies the selected base multiplier (i.e., 1.5) by the average period of time (i.e., 6 days) that similarly situated individuals take the medication to calculate a number of days (in this case, 9) between the prescription date and the therapeutic use date. Assuming the date that the prescription is being filled is Aug. 6, 2019, the information processing system 102 can use the result of its calculations to determine the therapeutic use date of Aug. 15, 2019.

### Other Variations

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for associating units of medication with a therapeutic use of the medication by an individual, the method comprising:
identifying prescription information associated with an individual and a prescription for a plurality of units of medication;
determining a therapeutic use date to be added to each of the plurality of units of the medication based at least in part on the prescription information, wherein the therapeutic use date is different from and later than a strict adherence date associated with the prescription, wherein the therapeutic use date is different from and earlier than an expiration date of the plurality of units of the medication; and
causing an apparatus to add the therapeutic use date to each of plurality of units of the medication.

2. The method of claim 1, wherein the therapeutic use date corresponds to a date after which consumption of any of the plurality of units of the medication will have no legitimate therapeutic value to the individual.

3. The method of claim 1, wherein the therapeutic use date corresponds to a date after which the amount of therapeutic value provided to the individual when consuming any of the plurality of units of the medication does not satisfy a threshold therapeutic value.

4. The method of claim 1, wherein the therapeutic use date corresponds to a date by which the individual should no longer be in possession of any of the plurality of units of the medication.

5. The method of claim 1, wherein the therapeutic use date corresponds to a date after which the individual should dispose of any unused ones of the plurality units of the medication.

6. The method of claim 1, wherein the therapeutic use date corresponds to a date after which the individual return any unused ones of the plurality units of the medication to a medication-return facility.

7. The method of claim 1, wherein the medication is at least one of a prescription opioid, central nervous system depressant, or stimulant.

8. The method of claim 1, wherein the prescription information comprises at least a number units of the medication in the prescription and a dosing schedule associated with the prescription, wherein the dosing schedule indicates an amount of time between doses of the medication.

9. The method of claim 8, wherein said determining the therapeutic use date comprises:
multiplying the number units of the medication in the prescription by the amount of time between doses of the medication to determine a minimum amount of time the prescription can be completed according to the dosing schedule;
selecting a base multiple based at least in part on the prescription information; and
multiplying the base multiplier by the minimum amount of time the prescription can be completed according to the dosing schedule.

10. The method of claim 9, wherein the therapeutic use date is extrapolated based at least in part on a date of the prescription and said multiplying the base multiplier by the minimum amount of time the prescription can be completed according to the dosing schedule.

11. The method of claim 9, wherein the base multiplier is an individual-specific base multiplier, the base multiplier being based at least in part on a historical use of the medication by the individual.

12. The method of claim 9, wherein the strict adherence date corresponds to a minimum amount of time the prescription can be completed according to the dosing schedule.

13. The method of claim 1, wherein each of the plurality of units of the medication comprises at least one of a pill, tablet, or capsule.

14. The method of claim 1, wherein the expiration date is based at least in part on at least one of a chemical efficacy of the prescribed medication.

15. A system for associating units of medication with a therapeutic use of the medication by an individual, the system comprising:
one or more processors configured to:
identify prescription information associated with an individual and a prescription for a plurality of units of medication;
determine therapeutic use date to be added to each of the plurality of units of the medication based at least in part on the prescription information, wherein the therapeutic use date is different from and later than a strict adherence date associated with the prescription, wherein the therapeutic use date is different from and earlier than an expiration date of the plurality of units of the medication; and
cause an apparatus to add the therapeutic use date to each of plurality of units of the medication.

16. The system of claim 15, wherein the therapeutic use date corresponds to a date after which consumption of any of the plurality of units of the medication will have no legitimate therapeutic value to the individual.

17. The method of claim 1, wherein the therapeutic use date corresponds to a date by which the individual should no longer be in possession of any of the plurality of units of the medication.

18. The system of claim 15, wherein to determine the therapeutic use date the one or more processors are further configured to:
multiply a number units of the medication in the prescription by an amount of time between doses of the medication to determine a minimum amount of time the prescription can be completed according to a dosing schedule;
select a base multiple based at least in part on the prescription information; and
multiply the base multiplier by the minimum amount of time the prescription can be completed according to the dosing schedule.

19. The system of claim 18, wherein the base multiplier is an individual-specific base multiplier, the base multiplier being based at least in part on a historical use of the medication by the individual.

20. The system of claim 15, wherein the strict adherence date corresponds to a minimum amount of time the prescription can be completed according to the dosing schedule.
